(19) United States
(12) Patent Application Publication
(10) Pub. No.: US 2026/0176219 A1
(43) Pub. Date: Jun. 25, 2026

(54) Dehydrogenation Process

(71) Applicants: Clariant International Ltd, Muttenz (CH); Lummus Technology LLC, Houston, TX (US)

(72) Inventors: Vladimir Fridman, Louisville, KY (US); Sunil Panditrao, Budd Lake, NJ (US); Prashant Sankarankandath, Louisville, KY (US)

(21) Appl. No.: 19/427,032

(22) Filed: Dec. 19, 2025

Related U.S. Application Data
(60) Provisional application No. 63/737,030, filed on Dec. 20, 2024.

(51) Int. Cl. C07C 5/32

(52) U.S. Cl. CPC C07C 5/322

(57) ABSTRACT

The present disclosure relates generally to processes and systems for dehydrogenating alkanes. The present disclosure relates specifically to processes and systems for dehydrogenating alkanes that include purging the one or more catalyst beds by flowing therethrough a first purge gas stream having no more than 5 vol % O₂ and in a first pressure reduction reducing the pressure in the dehydrogenation reactor to no more than 0.5 bar.

### BACKGROUND OF THE DISCLOSURE

### Field

The present disclosure relates to processes for alkane dehydrogenation, and systems for the same.

### Technical Background

Several endothermic hydrocarbon conversion processes are utilized in commercial operations. These processes include the CATOFIN cyclic fixed bed dehydrogenation process, the fluid bed paraffin dehydrogenation process, the fluid bed ethylbenzene dehydrogenation process, and fluid bed catalytic cracking process, among others. Because these processes are endothermic, heat must be consumed from the surroundings in order for the hydrocarbon conversion reaction to occur. In each of these processes, there is a conversion reaction that is promoted by contacting a hydrocarbon feed with a catalyst. In each of these processes, after a conversion cycle there is at least one reducing and/or oxidizing reaction that refreshes the catalyst for another conversion cycle. Often parallel reactors are used such that there is always at least one reactor that is in a conversion cycle. The heat needed for the endothermic reactions to occur is provided in part by combustion of coke and other undesirable side products that deposit on the catalyst during the conversion process. However, additional heat is typically needed; this is typically provided by hot air or steam that is fed into the catalyst bed from external sources between the hydrocarbon conversion cycles.

As an example, in the typical Houdry dehydrogenation process as taught in U.S. Pat. No. 2,419,997, an aliphatic hydrocarbon passes through a dehydrogenation catalyst bed. As the aliphatic hydrocarbon passes through the catalyst bed, the hydrocarbon is dehydrogenated to its complementary olefin. The olefin is then flushed from the catalyst bed, the catalyst is regenerated to remove deposits like coke by treatment with air, then reduced with hydrogen to provide active catalyst. This cycle is repeated. This dehydrogenation reaction is highly endothermic, and accordingly during the dehydrogenation step the temperature near the inlet of the catalyst bed (where the aliphatic hydrocarbon initially enters the catalyst bed) can decrease by as much as 100° C. This decrease in temperature causes a decrease in hydrocarbon conversion. In addition, during the dehydrogenation step, it is common for coke to form and deposit on the catalyst, further reducing the activity of the catalyst.

In U.S. Pat. No. 2,423,835, Houdry teaches that the catalyst bed temperature may be controlled within a temperature range suitable for the reactions without requiring an extraneous heating or cooling fluid to be circulated through or around the reaction chamber by including within the catalyst bed “inert” material capable of absorbing or storing up heat which can be subsequently released as required. In commercial practice for fixed bed reactors, this is typically achieved by using a physical mixture of a dehydrogenation catalyst and a granular alpha-aluminum “inert” material as the catalyst bed. However, this process still requires an external heat source.

In more recent years, Clariant has pioneered the use of a so-called “heat-generating material” in a dehydrogenation catalyst bed. The heat-generating material can be physically mixed in with the dehydrogenation catalyst, and is generally catalytically inert with respect to dehydrogenation and side reactions (e.g., cracking or coking). But, critically, it generates heat under oxidizing and/or reducing reaction conditions. Thus, during the regeneration and/or catalyst reducing steps, the heat-generating material can heat up the catalyst bed, i.e., including the dehydrogenation catalyst. Accordingly, while the dehydrogenation catalyst will cool down during the dehydrogenation step as a result of the endothermicity of the dehydrogenation reaction, the use of the heat-generating material can efficiently heat it back up during the regeneration and/or reduction steps of the cycle.

In all of these processes, the regeneration step is used to regenerate the catalyst by removing the coke that has deposited on the catalyst as well as to heat up the catalyst bed. In a typical regeneration step, the reactor is purged of hydrocarbons then air is flowed through the catalyst bed at temperatures of up to 700° C. Heat is provided to the bed by the hot air that passes through the bed and also by the exothermicity of the combustion of the coke deposits on the catalyst. During regeneration, the hot air flows from the inlet of the catalyst bed to the outlet. This regeneration cycle is normally relatively short, and the reactor is generally evacuated before moving onto reduction.

And in all processes, the reducing step acts to further heat the catalyst bed as well as put the metallic catalyst in a desired reduced state for hydrogenation. The degree of conversion of alkene to alkane in the conversion is dependent on both of these effects. When the reduction is more inefficient, the overall efficiency of the process can suffer.

There remains a challenge to provide improved dehydrogenation process that can provide better overall performance, especially as it relates to the reduction step.

### SUMMARY

In one aspect, the present disclosure provides for a process for the dehydrogenation of hydrocarbons, the process comprising:
providing a dehydrogenation reactor comprising one or more catalyst beds, each of the one or more catalyst beds comprising a dehydrogenation catalyst, and, optionally, a granular inert material; and
performing the following cycle of steps a plurality of times:
   regenerating the one or more catalyst beds by contacting the one or more catalyst beds with an oxygen-containing stream; then
   purging the one or more catalyst beds by flowing therethrough a first purge gas stream having no more than 5 vol % O₂ and reducing the pressure in the dehydrogenation reactor to no more than 0.5 bar; then
   reducing the one or more catalyst beds by flowing therethrough a reducing stream comprising hydrogen, at least part of the reduction being
   conducted at at least 0.9 bar of hydrogen partial pressure; then contacting the one or more catalyst beds with an alkane stream by flowing from an upstream direction to a downstream direction, to dehydrogenate the alkane stream to provide an alkene stream, the dehydrogenation being endothermic; then
   purging the one or more catalyst beds with a second purge gas stream (e.g., with a steam stream) to substantially remove hydrocarbon therefrom.

In another aspect, the present disclosure provides a system for the dehydrogenation of hydrocarbons, the system comprising:
a dehydrogenation reactor comprising one or more catalyst beds, each of the one or more catalyst beds comprising a dehydrogenation catalyst, and optionally a granular inter material and/or a heat-generating material, the heat-generating material being configured to generate heat in response to at least one of a reducing of the catalyst bed in which it is disposed and regeneration of the catalyst bed in which it is disposed;
a source of a reducing gas stream comprising hydrogen, operatively coupled to the one or more catalyst beds; and
a source of an alkane stream, operatively coupled to the at least one catalyst bed and configured to flow from an upstream direction to a downstream direction of the dehydrogenation reactor; and
a source of an oxygen-containing stream operatively coupled to the one or more catalyst beds, through an operative coupling to the dehydrogenation reactor;
a heater (e.g., an organic-burning heater) capable of emitting a first purge gas (e.g., a flue gas), operatively coupled to the one or more catalyst beds, through an operative coupling to the dehydrogenation reactor;
a vacuum pump, operatively coupled to the one or more catalyst beds, through an operative coupling to the dehydrogenation reactor.

Other aspects of the disclosure will be apparent to those skilled in the art in view of the description that follows.

### BRIEF DESCRIPTION OF THE DRAWINGS

FIG. 1 provides a schematic view of an embodiment of a system and process according to the disclosure.

FIG. 2 provides a partial schematic view of an embodiment of a system and process according to the disclosure.

### DETAILED DESCRIPTION

The present inventors have developed a process for improved catalyst reduction during alkane dehydrogenation.

The conventional light paraffin dehydrogenation process was developed by Houdry US, and is conventionally based on fixed-bed CrOₓ/Al₂O₃ catalysts operating in parallel, adiabatic, fixed-bed reactors operating in a cyclic mode. This process is described, for example, in U.S. Pat. Nos. 2,419,997 and 2,423,029, each of which is hereby incorporated herein by reference in its entirety. In typical operation, the cycle starts with the reduction of the catalyst bed by a mixture of H₂ and light hydrocarbons. Subsequently, dehydrogenation of an alkane feed takes place under vacuum. After dehydrogenation, the reactor and catalyst bed is steam purged to substantially remove hydrocarbons, and then regenerated and reheated by hot air. Finally, the reactor is evacuated to complete the cycle.

Regeneration is important, as it not only cleans deposits off of the dehydration catalyst, but it also provides heat that can be consumed by a subsequent endothermic dehydrogenation reaction. Importantly, the level of paraffin conversion has been found to depend on dehydrogenation catalyst temperature, and thus on the efficiency of the heat supply during regeneration. Heat can be supplied by at least two mechanisms, including the exothermic reaction of coke burning, and direct heat transfer from the heated air used in the regeneration. Heated air can be provided at a temperature up to 680° C. in some instances, and fuel gas may be added to the hot air in order to further increase the effective temperature in the reactor to 730° C. Heat is can also typically added to the catalyst in reducing step, by the exothermicity of the reduction of high oxidation state chromium.

Reduction of the catalyst is performed after the regeneration. The reducing step is an important step of the process because it not only puts the catalyst in the reduced state that is active during dehydrogenation, but also provides heat to catalyst bed that is used for endothermic dehydrogenation reaction. The degree of conversation of alkenes to alkanes in the dehydrogenation depends on both of these features of the reduction.

However, the present inventors have noted a problem in many conventional processes: presence of oxygen during the reduction and dehydrogenation steps. Any oxygen present during reduction can react with hydrogen and/or oxidize the catalyst, which can provide for a lower efficiency of reduction. And oxygen present during the dehydrogenation can react with the hydrocarbon reactants and products to make CO₂, representing an undesirable loss of useable carbon. The formation of CO₂ in the process can create problems downstream, including solidifying at lower temperatures and plugging up downstream pipes.

In the dehydrogenation process, there are three main sources of oxygen that are ideally eliminated from the reactor before the introduction of hydrocarbons. Both the heat-generating material and the oxidized catalyst after regeneration (e.g., Cr⁶⁺ oxide) can provide oxygen to the system. These can be addressed through effective reduction.

The third main source of oxygen is oxygen remaining in the catalyst bed left over from the regeneration step. While typical processes evacuate the catalyst bed to remove oxygen from the catalyst bed, the present inventors have determined that this can leave significant amounts of oxygen present, e.g., on the order of half of the oxygen present during the regeneration. This oxygen can remain present not only during reduction, lowering the efficiency of the reduction, but also into dehydrogenation, where it can react with the hydrocarbons to form CO₂.

The second and third sources of oxygen can be minimized by improving reduction before the dehydrogenation step. Unfortunately, the existing design of the dehydrogenation process only removes half of the oxygen present from the regeneration step. As such, that oxygen remains present during reduction, leading to reduction is conducted at lower partial pressure of hydrogen, thus decreasing the efficiency of reduction. With incomplete reduction of the heat-generating material and dehydrogenation catalyst, catalyst selectively decreases and CO₂ increases.

In order to address this issue, the present inventors have determined that it is desirable to be introduce a purge of the catalyst bed after regeneration. There are a number of ways to do this, as the person of ordinary skill in the art will appreciate. But it has been presently determined that in some embodiments a source of flue gas (or other low-oxygen containing gas) can be provided to provide the purge gas stream. This approach can flush oxygen from the system before catalyst reduction, and thus advantageously allow for improved reduction of the catalyst, reduced consumption of the reduction gas, and a more effective and efficient dehydrogenation step.

Accordingly, in one aspect, the present disclosure provides for a process for the dehydrogenation of hydrocarbons, the process comprising:
providing a dehydrogenation reactor comprising one or more catalyst beds, each of the one or more catalyst beds comprising a dehydrogenation catalyst, and, optionally, a granular inert material; and
performing the following cycle of steps a plurality of times:
   regenerating the one or more catalyst beds by contacting the one or more catalyst beds with an oxygen-containing stream; then
   purging the one or more catalyst beds by flowing therethrough a first purge gas stream having no more than 5 vol % O₂ and reducing the pressure in the dehydrogenation reactor to no more than 0.5 bar; then
   reducing the one or more catalyst beds by flowing therethrough a reducing stream comprising hydrogen, at least part of the reduction being conducted at at least 0.9 bar of hydrogen partial pressure; then
   contacting the one or more catalyst beds with an alkane stream by flowing from an upstream direction to a downstream direction, to dehydrogenate the alkane stream to provide an alkene stream, the dehydrogenation being endothermic; then
purging the one or more catalyst beds with a second purge gas stream (e.g., with a steam stream) to substantially remove hydrocarbon therefrom.

In another aspect, the present disclosure provides for a system for the dehydrogenation of hydrocarbons, the system comprising:
a dehydrogenation reactor comprising one or more catalyst beds, each of the one or more catalyst beds comprising a dehydrogenation catalyst, and optionally a granular inter material and/or a heat-generating material, the heat-generating material being configured to generate heat in response to at least one of a reducing of the catalyst bed in which it is disposed and regeneration of the catalyst bed in which it is disposed;
a source of a reducing gas stream comprising hydrogen, operatively coupled to the one or more catalyst beds; and
a source of an alkane stream, operatively coupled to the at least one catalyst bed and configured to flow from an upstream direction to a downstream direction of the dehydrogenation reactor; and
a source of an oxygen-containing stream operatively coupled to the one or more catalyst beds, through an operative coupling to the dehydrogenation reactor;
a heater (e.g., an organic burning heater) capable of emitting a first purge gas (e.g., a flue gas), operatively coupled to the one or more catalyst beds, through an operative coupling to the dehydrogenation reactor;
a vacuum pump, operatively coupled to the one or more catalyst beds, through an operative coupling to the dehydrogenation reactor.
   The system according to this aspect can be used, for example, to perform the methods described herein.

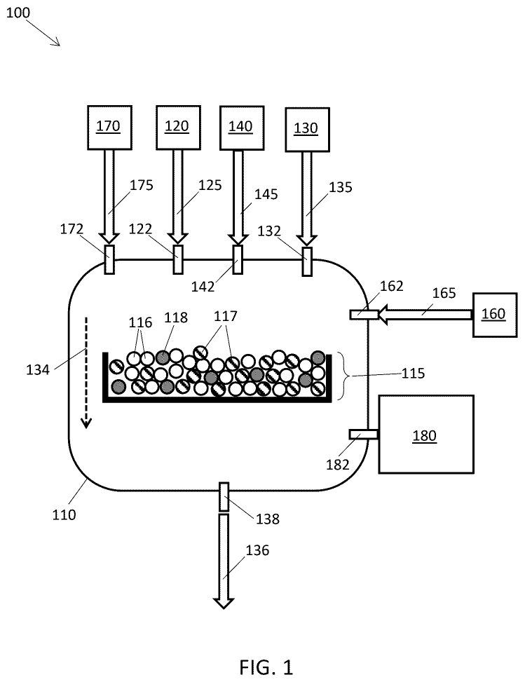

One example is shown in schematic view in FIG. 1. Here a system 100 for the dehydrogenation of hydrocarbons includes a dehydrogenation reactor 110, that includes one or more (here, one) catalyst beds 115. As used herein, a catalyst bed includes a dehydrogenation catalyst (e.g., 116 in FIG. 1) and can optionally include a granular inert material (e.g., 118 in FIG. 1) and/or a heat-generating material (e.g., 117 in FIG. 1). The heat-generating material is configured to generate heat in response to at least one of a reducing of the catalyst bed in which it is disposed and regeneration of the catalyst bed in which it is disposed. A source 120 of a reducing gas stream 125 is operatively coupled to the catalyst bed 115, here, through coupling to the reactor 110 at reducing gas inlet 122 thereof. A source 130 of an alkane stream 135 is operatively coupled to the catalyst bed 115, here, through coupling to the reactor 110 at alkane stream inlet 132 thereof. A source 140 of an oxygen-containing stream 145 is operatively coupled to the catalyst bed 115, here, through coupling to the reactor 110 at oxygen-containing stream inlet 142 thereof. A heater (e.g., an organic-burning heater) 160 capable of emitting a first purge gas stream 165 (e.g., a flue gas) is operatively coupled to the catalyst bed 115, here, through coupling to reactor 110 at a first purge gas stream inlet 162. A vacuum pump 180 operatively coupled to the catalyst bed 115, here, through coupling to reactor 110 at vacuum inlet 182. In various embodiments, and as shown in FIG. 1, a source 170 of a second purge gas stream 175 (e.g., a steam stream) is operatively coupled to the catalyst bed 115, here through coupling to the reactor 110 at second purge gas stream inlet 172 thereof.

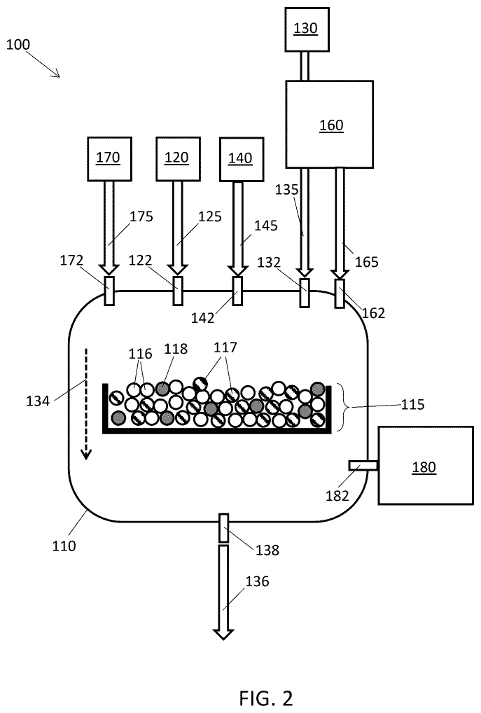

In various embodiments of the system as described herein, the heater (e.g., an organic burning heater) is configured to heat the alkane stream and to provide a first purge gas stream to purge the one or more catalyst beds. For example, in various embodiments as shown in FIG. 2, the heater (e.g., an organic burning heater) 160 is configured to heat the alkane stream 135 and to provide a first purge gas stream 165 to purge the catalyst bed 115.

In one embodiment of process for dehydrogenating hydrocarbons of the disclosure, a dehydrogenation reactor 110 comprising one or more (here, one) catalyst beds 115, each comprising a dehydrogenation catalyst 116, and optionally a granular inert material 118 and/or a heat-generating material 117. The process includes performing a cycle of steps a plurality of times. The cycle includes, in a regeneration step, the one or more catalyst beds 115 are contacted with an oxygen-containing stream 145 by introducing the oxygen-containing stream to the dehydrogenation reactor (here, through oxygen-containing stream inlet 142). This regeneration step is performed at a temperature sufficient to regenerate the one or more catalyst beds, i.e., by removing a substantial degree of coke and other deposits, and by substantially increasing the temperature of the one or more catalyst beds. Then, in a purging step (e.g., a first purging step), the one or more catalyst beds 115 are purged by flowing therethrough a first purge gas stream 165 having no more than 5 vol % O₂, here, by introduction through first purge gas stream inlet 162, and reducing the pressure in the dehydrogenation reactor to no more than 0.5 bar, here by contact with vacuum 180 through vacuum inlet 182. Then, in a reducing step, reducing the one or more (here, one) catalyst beds 115 by flowing therethrough a reducing stream 125 comprising hydrogen, here, by introduction through reducing gas inlet 122. Then, in a dehydrogenation step, the one or more (here, one) catalyst beds 115 are contacted with an alkane stream, here, by introduction through alkane stream inlet 132 by flowing from an upstream direction to a downstream direction (as indicated by arrow 134), to dehydrogenate the alkane stream to provide an alkene stream 136, which can be removed from the reactor at outlet 138 thereof. Notably, the dehydrogenation is endothermic. As described above, the endothermicity of the dehydrogenation can cause the temperature of the catalyst bed to decrease, which undesirably affects dehydrogenation catalyst performance. Coke and other side reaction products can also collect on the dehydrogenation catalyst, which also undesirably affects dehydrogenation catalyst performance. Accordingly, after the catalyst bed is purged to substantially remove hydrocarbons (e.g., with a second purge gas stream 175 from second purge gas stream source 170 admitted to the reactor at steam stream inlet 172, the next step is a regeneration step, and the cycle repeats.

As described above, the cycle includes regenerating the one or more catalyst beds by contacting the one or more catalyst beds with an oxygen-containing stream. As used herein, an “oxygen-containing stream” is defined as the entirety of the oxygen-containing gas input to the dehydrogenation reactor during the regeneration. As would be understood by a person of ordinary skill in the art, the entire oxygen-containing stream can be provided to the reactor at a single inlet. For example, in the embodiment of FIG. 1, oxygen-containing stream 145 is introduced to the dehydrogenation reactor at oxygen-containing stream inlet 142. The oxygen-containing stream can be provided by the person of ordinary skill in the art in view of the teachings herein and of the state of the art. In various embodiments, the oxygen-containing gas is substantially made of air. But other oxygen-containing streams can be used. Desirably, the oxygen-containing stream includes at least 5 wt % oxygen, e.g., at least 10 wt %.

As the person of ordinary skill in the art will appreciate, the oxygen-containing stream used in the regeneration is desirably rather hot, so as to provide for substantially complete combustion of coke and other deposits, and to provide for desirable heating of the dehydrogenation catalyst. For example, in various embodiments as otherwise described herein, the oxygen-containing stream during at least one stage of the regeneration, has a temperature in the range of 500° C. to 800° C. (e.g., in the range 550° C. to 800° C., or 600° C. to 800° C., or 500° C. to 750° C., or 500° C. to 700° C.).

While the oxygen-containing gas can itself be provided at high temperature, it can also include a fuel gas that can combust in the one or more catalyst beds and provide additional heat energy. In some embodiments as described herein, the oxygen-containing stream further comprises a fuel gas.

As described above, the present inventors have found that including a purging step between regenerating and reducing the one or more catalyst beds can advantageously remove oxygen from the catalyst bed to provide improved catalyst reduction and efficiency. For example, in some embodiments as described herein, the purging and pressure reduction are performed to remove at least 70% of the oxygen from the catalyst bed (e.g., at least 80%). For example, in various embodiments, the purging pressure reduction are performed to remove at least 90%, or at least 95%, of the oxygen from the catalyst bed.

As used herein, a “first purge gas stream” is defined as the entirety of the low oxygen-containing gas input to the dehydrogenation reactor during the purging step. As would be understood by a person of ordinary skill in the art, the first purge gas stream can be provided to the reactor at a single inlet. For example, in the embodiment of FIG. 1, first purge gas stream 165 is introduced to the dehydrogenation reactor at first purge gas stream inlet 162. The first purge gas stream is advantageously low in oxygen gas. Accordingly, the purging step as described herein includes purging the one or more catalyst beds by flowing therethrough a first purge gas stream having no more than 5 vol % oxygen gas (i.e., O₂). In some embodiment as described herein, the first purge gas stream comprises no more than 4 vol % oxygen gas (e.g., no more than 3 vol % oxygen gas). For example, in various embodiments, the first purge gas stream comprises no more than 2 vol %, no more than 1 vol %, or no more than 0.5 vol %. In various embodiments as described herein, the first gas stream comprises 1-5 vol %, or 1-4 vol %, or 1-3 vol %, or 2-5 vol %, or 2-4 vol %, oxygen gas.

It is also advantageous to have low amounts of reactive gases (i.e., those present in the dehydrogenation process) in the first purge gas stream. For example, in various embodiments, the first purge gas stream comprises no more than 5 vol %, or no more than 2 vol %, or no more than 0.5 vol %, hydrogen. In some embodiments as described, the first purge gas stream comprises no more than 5 vol % hydrocarbon, e.g., no more than 2 vol %, or no more than 0.5 vol %.

Accordingly, the first purge stream will typically include a substantial amount of inert gas. By including a substantial amount of inert gas in the first purge stream, oxygen and other gases present in the dehydrogenation reactor after the regenerating step can be removed from the reactor and provide an inert atmosphere before the reducing step. In various embodiments as described herein, the first purge gas stream comprises at least 50 vol %, or at least 60 vol %, or at least 70 vol %, or at least 80 vol % inert gas. The identity of the inert gas is not particularly limited. For example, the inert gas may be selected from nitrogen or argon. In some embodiments as described herein, the first purge gas stream comprises at least 50 vol % nitrogen gas (e.g., at least 60 vol %, or at least 70 vol %, or at least 80 vol %).

Other gases may also be present in the first purge gas stream. For example, in some embodiments, the first purge gas stream comprises up to 15 vol % CO₂ (e.g., up to 10 vol %, or up to 12 vol %). In some embodiments as described herein, the first purge gas stream comprises up to 15 vol % H₂O (e.g., up to 12 vol %, or up to 10 vol %, or from 5-12 vol %, or 3-10 vol %). In some embodiments as described herein, the first purge gas stream comprises up to 3 vol % CO (e.g., up to 2%, or from 0.5-3%, or 0.5-2 vol %).

The source of the first purge gas stream is not particularly limited. For example, in some embodiments, the first purge gas stream may be provided from a source external to the dehydrogenation process (e.g., a stream of low-oxygen containing gas). In other embodiments, the first purge gas stream may be provided from a source integrated with the dehydrogenation process. Advantageously, the present inventors have found that flue gas can be an appropriate source of the first purge gas stream. Accordingly, in some embodiments as described herein, the first purge gas comprises a flue gas that comprises products of combustion of hydrocarbons (i.e., CO₂ and H₂O). As shown in FIGS. 1 and 2, a heater (e.g., an organic-burning heater) used in the dehydrogenation process can provide a flue gas stream. In some embodiments as described herein, the flue gas is provided by an organic-burning heater having a flue output operatively coupled to one or more catalyst beds. In some embodiments as described herein, the process and system includes heating the alkane stream with the organic-burning heater.

The flowing of the first purge gas stream may be conducted for a time sufficient to evacuate oxygen from dehydrogenation reactor. In some embodiments as described herein, the flowing of the first purge gas stream is performed for a time of at least 20 seconds, e.g., at least 30 seconds or at least 40 seconds. For example, in various embodiments, the flowing of the first purge gas stream is performed for a time in the range of 20-90 seconds, e.g., 20-75 seconds, or 20-60 seconds, or 30-90 seconds, or 30-75 seconds, or 30-60 seconds, or 40-90 seconds, or 40-75 seconds, or 40-60 seconds. Of course, a desired time of flowing of the first purge gas stream will depend on other factors, for example, pressure and flow rate of purge gas, and the person of ordinary skill in the art can, based on the present disclosure, adopt a desirable time for the flowing of the purge gas stream.

As described above, the process includes purging the one or more catalyst bed by flowing therethrough the first purge gas stream and in a first pressure reduction reducing the pressure in the dehydrogenation reactor to no more than 0.5 bar. In some embodiments as described herein, the first pressure reduction begins at a pressure inside the dehydrogenation reactor of at least 1 bar and provides a pressure of no more than 0.5 bar. For example, in various embodiments, the first pressure reduction begins at a pressure inside the dehydrogenation reactor of at least 1.5 bar, e.g., at least 1.7 bar, or 1.9 bar. In various embodiments, the first pressure reduction provides a pressure no more than 0.3 bar, e.g., no more than 0.2 bar. In some embodiments as described herein, the first pressure reduction provides a pressure inside the dehydrogenation reactor of at least 0.05 bar, e.g., at least 0.1 bar. Reducing the pressure in the dehydrogenation reactor during the first pressure reduction may be conducted as show in FIG. 1, here by contact with vacuum 180 through vacuum inlet 182.

In various embodiments as described herein, the first pressure reduction is performed for a time of at least 20 seconds, e.g., at least 30 seconds or at least 40 seconds. For example, in various embodiments, the first pressure reduction is performed for a time in the range of 20-90 seconds, e.g., 20-75 seconds, or 20-60 seconds, or 30-90 seconds, or 30-75 seconds, or 30-60 seconds, or 40-90 seconds, or 40-75 seconds, or 40-60 seconds. Of course, a desired time of the first pressure reduction will depend on other factors, and the person of ordinary skill in the art can, based on the present disclosure, adopt a desirable time for the first pressure reduction.

As described above, the purging includes flowing the first purge gas and the first pressure reduction. These can be performed in any desirable order. For example, in some embodiments, the first pressure reduction is performed after the flow of the first purge gas. In some embodiments, the first pressure reduction overlaps with the flow of the first purge gas, e.g., entirely overlaps.

As described above, the process for the dehydrogenation of hydrocarbons also includes reducing the one or more catalyst beds by flowing therethrough a reducing stream comprising hydrogen. As used herein, a “reducing stream” is defined as the entirety of the reducing gas input to the dehydrogenation reactor during the reducing step. As would be understood by a person of ordinary skill in the art, the entire reducing stream can be provided to the reactor at a single inlet. For example, in the embodiment of FIG. 1, reducing stream 125 is introduced to the dehydrogenation reactor at reducing stream inlet 122. The reducing stream for the reduction of the catalyst can be provided as is conventional in the art. For example, in various embodiments as otherwise described herein, the reducing gas stream comprises hydrogen gas, optionally provided with an inert gas such as nitrogen. In some embodiments as described herein, the reducing stream of the reducing step further comprises hydrocarbons (e.g., CH₄, C₂H₆, and/or C₂H₄). In some embodiments as described herein, the reducing stream of the reducing step further comprises CO. In some embodiments as described herein, the reducing stream of the reducing strep further comprises CO₂. In some embodiments as described herein, the reducing stream is mostly hydrogen gas. For example, in various embodiments as described herein, reducing stream of the reducing step comprises at least 50 vol %, at least 60 vol %, or at least 70 vol % hydrogen gas. In some embodiments as described herein, the reducing stream of the reducing step substantially comprises hydrogen gas. For example, in various embodiments, the reducing stream comprises at least 80 vol %, at least 85 vol %, at least 90 vol %, or at least 95 vol %, hydrogen gas.

As described above, during the reducing step of the process and systems as described herein, at least a part of the reduction is conducted at at least 0.9 bar of hydrogen partial pressure. In some embodiments as described herein, at least part of the reduction is conducted at at least 1 bar of hydrogen partial pressure, or at at least 1.1 bar of hydrogen partial pressure, or at at least 1.2 bar of hydrogen partial pressure.

As noted by the present inventors, it is advantageous for the reducing step to be conducted with a reducing stream that is mostly hydrogen gas. However, as described above, other gases may be present in the reducing stream (although to a lesser amount than hydrogen). As such, in some embodiments as described herein, the total pressure in the dehydrogenation reactor during the reducing step is greater than the hydrogen partial pressure. In some embodiments as described herein, at least part of the reducing step is performed at a pressure in the dehydrogenation reactor of at least 1.1 bar. For example, in various embodiments, at least part of the reducing step is performed at a pressure in the dehydrogenation reactor of at least 1.3 bar, e.g., at least 1.5 bar. For example, in various embodiments, at least part of the reducing step is performed at a pressure in the dehydrogenation reactor in the range of 1.1-2 bar, e.g., 1.1-1.8 bar, or 1.3-2 bar, or 1.3-1.8 bar, or 1.5-2 bar, or 1.5-1.8 bar.

Here, the present inventors have found that the processes and systems described herein can advantageously improve the reducing step efficiency and efficacy. For example, in some embodiments as described herein, the amount of hydrogen present in the reducing stream decreases during the reducing step. In various embodiments, the amount of hydrogen present in the reducing stream decreases by at least 40%, at least 50%, at least 60%, or at least 70%, during the reducing step. As would be understood by the person of ordinary skill in the art, the decrease of the amount of hydrogen present in the reducing stream can be measured by comparing the composition of the reducing stream prior to and post contact with the one or more catalyst beds. Additionally, with increased hydrogen consumption, more complete reduction of the one or more catalysts can be accomplished. For example, in some embodiments as described herein, the reducing step results in at least 60% reduction of the gas-accessible surface area of the one or more catalyst beds, e.g. at least 70%, or at least 80%.

The reduction can be performed for a variety of times; the person of ordinary skill in the art can, based on the present disclosure, select a reduction time to provide a desired degree of reduction and catalyst heating. In various embodiments as described herein, the reduction is conducted for at least 10 seconds, e.g., at least 20 seconds or at least 30 seconds. In some embodiments, the reduction is conducted for a time in the range of 10-120 seconds, e.g., 10-90 seconds, or 20-120 seconds, or 20-90 seconds, or 30-120 seconds, or 30-90 seconds.

In various embodiments as described herein, the reducing step further comprises a second pressure reduction, e.g., to reduce the pressure in the dehydrogenation reactor to no more than 0.5 bar. In some embodiments as described herein, the second pressure reduction provides a pressure inside the dehydrogenation reactor of no more than 0.4 bar. For example, in various embodiments, the second pressure reduction provides a pressure inside the dehydrogenation reactor of no more than 0.3 bar, or no more than 0.2 bar. In some embodiments as described herein, the second pressure reduction provides a pressure inside the dehydrogenation reactor of at least 0.1 bar. In some embodiments as described herein, the second pressure reduction begins at a pressure of at least 1.1 bar (e.g., at least 1.3 bar, or at least 1.5 bar) and provides a pressure no more than 0.3 bar (e.g., no more than 0.2 bar). Reducing the pressure in the dehydrogenation reactor during the second pressure reduction step may be conducted as show in FIG. 1, here by contact with vacuum 180 through vacuum inlet 182.

In various embodiments as described herein, the second pressure reduction is performed for at least 10 seconds, e.g., at least 20 seconds, or at least 30 seconds. The second pressure reduction can be performed, e.g., for a time in the range of 10-90 seconds, e.g., 10-75 seconds, or 10-60 seconds, or 20-90 seconds, or 20-75 seconds, or 20-60 seconds, or 30-90 seconds, or 30-75 seconds, or 30-60 seconds. But the person of ordinary skill in the art will appreciate that other times are possible.

The second pressure reduction can be performed while the flow of reducing gas continues through the catalyst bed.

As described above, the catalyst reduction can include a second pressure reduction; in such cases, in various embodiments as described herein, the duration of the reduction (including the second pressure reduction) is no more than 300 seconds (e.g. no more than 240 seconds, no more than 180 seconds). In some embodiments as described herein, the duration from the beginning of the reducing step to the end of the second pressure reduction step is at least 30 seconds (e.g. at least 60 seconds). In some embodiments as described herein, the duration of the reduction is in the range of 30-300 seconds, e.g., in the range of 60-300 seconds, or 30-240 seconds, or 60-240 seconds, or 30-180 seconds, or 60-180 seconds.

After the reducing step, the processes as described herein including contacting the one or more catalyst beds with an alkane stream by flowing from an upstream direction to a downstream direction, to dehydrogenate the alkane stream to provide an alkene stream, the dehydrogenation being endothermic.

As used herein, an “alkane stream” is defined as the entirety of the alkane-containing gas input to the dehydrogenation reactor during the dehydrogenation. As would be understood by a person of ordinary skill in the art, the entire alkane stream can be provided to the reactor at a single inlet. For example, in the embodiment of FIG. 1, alkane stream 135 is introduced to the dehydrogenation reactor at alkane stream inlet 132. The alkane stream can be provided by the person of ordinary skill in the art in view of the teachings herein and of the state of the art. In various embodiments as otherwise described herein, the alkane stream comprises at least 50 wt % C₂-C₆ alkanes, as a percentage to total hydrocarbons (e.g., at least 60 wt %, or at least 75 wt %, or at least 85 wt %, or at least 90 wt %). For example, in particular embodiments, the alkane stream comprises at least 50 wt % C₂-C₄ alkanes, as a percentage to total hydrocarbons (e.g., at least 60 wt %, or at least 75 wt %, or at least 85 wt %, or at least 90 wt %). In various embodiments as described herein, the alkane stream comprises less than 10 wt % (e.g., less than 5 wt %, or less than 1 wt %) of high-coking hydrocarbons. The person of ordinary skill in the art will appreciate that a wide variety of alkane streams, including, for example, those described in the references described above.

As described above, the process includes contacting the one or more catalyst beds with an alkane stream by flowing from an upstream direction to a downstream direction, to dehydrogenate the alkane stream to provide an alkene stream, the dehydrogenation being endothermic. In some embodiments as described herein, the dehydrogenation step is performed at a pressure or pressures between 0.05 and 3.0 bar, e.g. 0.1 bar, or 0.2 bar, or 0.5 bar, or 1.0 bar, or 1.5 bar, or 2.0 bar, or 2.5 bar, or 3.0 bar, or between 0.1 and 3.0 bar, or between 0.2 and 3.0 bar, or between 0.5 and 3.0 bar, or between 0.1 and 2.5 bar, or between 0.2 and 2.5 bar, or between 0.5 and 2.5 bar, or between 0.1 and 2.0 bar.

After the dehydrogenation step, process continuing with purging the one or more catalyst bed with a second purge gas stream to substantially remove hydrocarbons therefrom. As used herein, a “second purge gas stream” is defined as the entirety of a purge gas input to the dehydrogenation reactor during the regeneration. As would be understood by a person of ordinary skill in the art, the entire second purge gas stream can be provided to the reactor at a single inlet. For example, in the embodiment of FIG. 1, second purge gas stream 175 is introduced to the dehydrogenation reactor at second purge gas stream inlet 172. The second purge gas stream can be provided by the person of ordinary skill in the art in view of the teachings herein and of the state of the art. In some embodiments as described herein, the second purge gas stream comprises steam. In some embodiments as described herein, the second purge gas stream is a steam stream. After the catalyst bed is purged to substantially remove hydrocarbons reactor, the next step is a regeneration step, and the cycle begins again. Accordingly, in various embodiments as described herein, the one or more catalyst beds operate in a cyclic redox mode.

As described, the dehydrogenation rector comprises one or more catalyst beds, each comprising a dehydrogenation catalyst, and optionally, a granular inert material.

Various dehydrogenation catalysts are known in the art. For example, in various embodiments as otherwise described herein, the dehydrogenation catalyst(s) of the one or more catalyst beds comprises chromium, e.g., in the form of an oxide. For example, in various embodiments, the chromium oxide is present in an amount of 5 wt % to 40 wt % of the dehydrogenation catalyst, calculated as Cr₂O₃. In various embodiments, the catalyst may be a supported catalyst. For example, in various embodiments, the dehydrogenation catalyst comprises an alumina support. In various embodiments, the dehydrogenation catalyst is produced by impregnation, co-mixing, or sol-gel methods. Particular dehydrogenation catalysts include those having chromium (15-30 wt % calculated as Cr₂O₃), sodium (0.1-1.5 wt %, calculated as oxide), potassium (0.1-3 wt % calculated as oxide), zirconium (0.1-2 wt % calculated as ZrO₂) and magnesium (0.3-1 wt % calculated as oxide), all on an alumina carrier.

But other dehydrogenation catalysts are known for use in cyclic dehydrogenation processes, and the person of ordinary skill in the art, based on the disclosure herein and the state of the art in cyclic dehydrogenations, can adapt these catalysts for use in the processes described herein. For example, U.S. Patent Application Publication no. 2023/0090285 a particulate dehydrogenation catalyst comprising a primary species P1 selected from Ga, In, TI, Ge, Sn, Pb, and any mixture thereof as an active metal, disposed on a support. Such dehydrogenation catalysts can be used in the processes described herein.

As described above, the one or more catalyst beds can also include, in addition to the dehydrogenation catalyst, a granular inert material, such as an alumina material, e.g., a granular alpha-alumina. Of course, other inert materials can be used. For example, in some embodiments as described herein, the granular inter material may be a mixture or silica and alumina, or zirconia and alumina, or titania and alumina, in any proportions. The inert can act as a heat sink and thus can help to maintain heat in the catalyst bed by providing thermal mass.

In some embodiments as described herein, the one or more catalyst beds further comprise a heat generating material. As described herein, at least one of the reducing and/or regenerating steps of the cycle causes the heat-generating material to generate heat. In order to improve the heat input and distribution to the catalyst bed, a heat-generating material can be used. As described above, the heat-generating material is generally catalytically inert with respect to dehydrogenation and side reactions, such as cracking or coking, but, critically, generates heat upon being exposed to oxidizing and/or reducing oxidizing reaction conditions. The heat-generating material can be mixed with the dehydrogenation catalyst, and, during the regeneration and/or reducing steps, heat up to provide heat to the dehydrogenation catalyst. Thus, the heat-generating material can help maintain desirably high catalyst bed temperatures over multiple cycles. It has been found that the use of heat-generating material can increase yields and save energy while simultaneously reducing emissions.

Various heat-generating materials are known in the art. For example, in various embodiments as otherwise described herein, the heat-generating material comprises a metal at a concentration of 2 wt % to 40 wt % of the total heat-generating material. In various embodiments, the metal of the heat-generating material comprises at least one of copper, chromium, molybdenum, vanadium, cerium, yttrium, scandium, tungsten, manganese, iron, cobalt, nickel, silver, or bismuth. The metal of the heat-generating material can be provided as metal, oxide, or any combination thereof. In some embodiments, the heat-generating material further comprises a promoter such as an alkali metal, an alkaline earth metal, or zirconium. Further details regarding heat-generating materials can be found in U.S. Pat. Nos. 7,622,623, 7,973,207, and 8,188,328, and U.S. Patent Application Publication no. 2023/0090285, each of which is hereby incorporated herein by reference in its entirety, for example, for teachings regarding heat-generating materials and cyclic dehydrogenation processes using the same. The person of ordinary skill in the art can adapt the heat-generating materials described herein for use in the processes and systems described here. One particular heat-generating material includes calcium (5-25 wt % calculated as oxide), magnesium (0.2-2 wt %, calculated as oxide), sodium (0.2-2 wt %, calculated as oxide), potassium (0.2-2 wt %, calculated as oxide), manganese (0.1-10 wt %, calculated as oxide), and copper (2-25 wt % calculated as CuO). Various examples of heat-generating materials include copper oxide, copper aluminate, calcium sulfate, copper sulfate, zinc oxide, nickel oxide, iron oxide, tin oxide, cobalt oxide, vanadium oxide, lanthanum oxide, cerium oxide and manganese oxide, which can be supported on an appropriate support. Another particular example of a heat-generating material is copper oxide supported on alumina, in which the copper oxide comprises at least about 8 wt % of the heat-generating inert component.

Accordingly, in various embodiments as otherwise described herein, one or more of (e.g., each of) the one or more catalyst beds includes heat-generating material in an amount that is at least 40 wt % of the combined amount of heat-generating material and dehydrogenation catalyst, e.g., at least 45 wt %, or at least 50 wt %. In various embodiments, one or more of (e.g., each of) the one or more catalyst beds includes heat-generating material in an amount in the range of 40-70 wt % of the combined amount of heat-generating material and dehydrogenation catalyst, e.g., 40-65 wt %, or 40-60 wt %, or 40-55 wt %, or 45-70 wt %, or 45-65 wt %, or 45-60 wt %, or 50-70 wt %, or 50-65 wt %.

While certain desirable relative amounts of heat-generating material in the one or more catalyst beds are described above, the person of ordinary skill in the art will appreciate that other amounts can be used. For example, in various embodiments, one or more of the one or more catalyst beds includes 15-70 wt % of dehydrogenation catalyst, e.g., 15-50 wt %, or 15-40 wt %, or 15-30 wt % (i.e., as a fraction of total amount of dehydrogenation catalyst, heat-generating material, and inert). In various embodiments, one or more of the one or more catalyst beds includes 15-70 wt %, of heat-generating material, e.g., 15-65 wt %, or 15-60 wt %, or 15-55 wt %, or 15-50 wt %, or 15-45 wt %, or 15-40 wt %, or 15-35 wt %, or 15-30 wt %, or 25-70 wt %, or 25-65 wt %, or 55-60 wt %, or 25-55 wt %, or 25-50 wt %, or 25-45 wt %, or 25-40 wt %, or 35-70 wt %, or 35-65 wt %, or 35-60 wt %, or 35-55 wt %, or 35-50 wt % (i.e., as a fraction of total amount of dehydrogenation catalyst, heat-generating material, and inert). In various embodiments, one or more of the one or more catalyst beds includes up to 70 wt % inert, e.g., up to 65 wt %, or up to 60 wt %, or up to 55 wt %, or up to 50 wt %, or up to 45 wt %, or up to 40 wt %, or up to 35 wt %, or up to 30 wt % (i.e., as a fraction of total amount of dehydrogenation catalyst, heat-generating material, and inert).

The person of ordinary skill in the art can otherwise perform the cyclic dehydrogenation processes and adapt the systems therefor using their skill and judgment, based on the disclosure herein. The person of ordinary skill in the art can apply any applicable embodiment of any cyclic dehydrogenation process or system known in the art, including those described in each of the following disclosures: U.S. Pat. Nos. 2,419,997, 2,423,029, 7,622,623, 7,973,207, and 8,188,328, and U.S. Patent Application Publication no. 2023/0090285.

The person of ordinary skill in the art can provide reaction conditions based on the particular system used, in view of the state of the art, including reaction conditions described in any embodiment of the references identified above. For example, in various embodiments as described herein, the dehydrogenation reactor operates at a temperature in the range of 500-1050° C., or 500-1000° C., or 500-950° C., or 500-900° C., or 500-850° C., or 500-800° C., or 500-750° C., or 500-700° C., or 500-650° C., or 500-600° C. In various embodiments as described herein, the dehydrogenation reactor operates at a temperature in the range of 500-700° C., or 520-700° C., or 540-700° C., or 560-700° C., or 580-700° C., or 600-700° C., or 500-680° C., or 500-660° C., or 500-640° C., or 500-620° C., or 500-600° C., or 520-680° C.

In some embodiments as described herein, the alkane stream is supplied to the one or more catalyst beds at a liquid hourly space velocity in the range of 0.1-4 per hour, e.g., 0.1-3 per hour, or 0.5-4 per hour, or 0.5-3 per hour, or 1-4 per hour, or 1-3 per hour.

In particular embodiments, the dehydrogenation is performed at a dehydrogenation temperature in the range of 520-680° C., a liquid hourly space velocity in the range of 0.8-2.5 h⁻¹, and a pressure in the range of 0.2-2 bar. Of course, the person of ordinary skill in the art will appreciate that a wide variety of reaction conditions can be applied.

Various exemplary embodiments of the disclosure include, but are not limited to the enumerated embodiments listed below, which can be combined in any number and in any combination that is not technically or logically inconsistent.

Embodiment 1. A process for the dehydrogenation of hydrocarbons, the process comprising:
providing a dehydrogenation reactor comprising one or more catalyst beds, each of the one or more catalyst beds comprising a dehydrogenation catalyst, and, optionally, a granular inert material; and
performing the following cycle of steps a plurality of times:
   regenerating the one or more catalyst beds by contacting the one or more catalyst beds with an oxygen-containing stream; then
   purging the one or more catalyst beds by flowing therethrough a first purge gas stream having no more than 5 vol % O₂ and in a first pressure reduction reducing the pressure in the dehydrogenation reactor to no more than 0.5 bar; then
   reducing the one or more catalyst beds by flowing therethrough a reducing stream comprising hydrogen, at least part of the reduction being conducted at at least 0.9 bar of hydrogen partial pressure; then
   contacting the one or more catalyst beds with an alkane stream by flowing from an upstream direction to a downstream direction, to dehydrogenate the alkane stream to provide an alkene stream, the dehydrogenation being endothermic; then
   purging the one or more catalyst beds with a second purge gas stream (e.g., with a steam stream) to substantially remove hydrocarbon therefrom.
      Embodiment 2. The process of embodiment 1, wherein the purging and pressure reduction are performed to remove at least 70% of the oxygen from the catalyst bed, e.g., at least 80%.
      Embodiment 3. The process of embodiment 1, wherein the purging and pressure reduction are performed to remove at least 90% of the oxygen from the catalyst bed, e.g., at least 95%.
      Embodiment 4. The process of embodiment 1 or embodiment 2, wherein the first purge gas stream comprises no more than 4 vol % oxygen gas (e.g., no more than 3%).
      Embodiment 5. The process of embodiment 1 or embodiment 2, wherein the first purge gas stream comprises 1-5 vol % oxygen gas (e.g. 1-4 vol %, or 1-3 vol %, or 2-5 vol %, or 2-4% vol %).
      Embodiment 6. The process of embodiment 1 or embodiment 2, wherein the first purge gas stream comprises no more than 2 vol % oxygen gas, e.g., no more than 1 vol %, or no more than 0.5 vol %.
      Embodiment 7. The process of any of embodiments 1-6, wherein the first purge gas stream comprises no more than 5 vol % hydrogen, e.g., no more than 2 vol %, or no more than 0.5 vol %.
      Embodiment 8. The process of any of embodiments 1-6, wherein the first purge gas stream comprises no more than 5 vol % hydrocarbon, e.g., no more than 2 vol %, or no more than 0.5 vol %.
      Embodiment 9. The process of any of embodiments 1-8, wherein the first purge gas comprises at least 50 vol % inert gas (e.g., at least 60 vol %, or at least 70 vol %, or at least 80 vol %).
      Embodiment 10. The process of any of embodiments 1-8, wherein the first purge gas comprises at least 50 vol % nitrogen gas (e.g., at least 60 vol %, or at least 70 vol %, or at least 80 vol %).
      Embodiment 11. The process of any of embodiments 1-10, wherein the first purge gas comprises up to 15 vol % CO₂ (e.g., up to 10 vol %, or up to 12 vol %).
      Embodiment 12. The process of any of embodiments 1-11, wherein the first purge gas comprises up to 15 vol % H₂O (e.g., up to 12 vol %, or up to 10 vol %, or 5-12 vol %, or 3-10 vol %).
      Embodiment 13. The process of any of embodiments 1-12, wherein the first purge gas comprises up to 3 vol % CO (e.g., up to 2%, such as 0.5-3 vol %, or 0.5-2 vol %).
      Embodiment 14. The process of any of embodiments 1-13, wherein first purge gas comprises a flue gas that comprises products of combustion of hydrocarbons.
      Embodiment 15. The process of embodiment 14, wherein the flue gas is provided by an organic-burning heater having a flue output operatively coupled to the one or more catalyst beds.
      Embodiment 16. The process of embodiment 15, further comprising heating the alkane stream with the organic-burning heater.
      Embodiment 17. The process of any of embodiments 1-16, wherein the flowing of the first purge gas stream is performed for a time of at least 20 seconds, e.g., at least 30 seconds or at least 40 seconds.
      Embodiment 18. The process of any of embodiments 1-16, wherein when the flowing of the first purge gas stream is performed for a time in the range of 20-90 seconds, e.g., 20-75 seconds, or 20-60 seconds, or 30-90 seconds, or 30-75 seconds, or 30-60 seconds, or 40-90 seconds, or 40-75 seconds, or 40-60 seconds.
      Embodiment 19. The process of any of embodiments 1-18, wherein the first pressure reduction begins at a pressure inside the dehydrogenation reactor of at least 1 bar and provides a pressure of no more than 0.5 bar.
      Embodiment 20. The process of any of embodiments 1-19, wherein the first pressure reduction begins at a pressure inside the dehydrogenation reactor of at least 1.5 bar, e.g., at least 1.7 bar, or at least 1.9 bar.
      Embodiment 21. The process of any of embodiments 1-19, wherein the first pressure reduction provides a pressure inside the dehydrogenation reactor of no more than 0.3 bar, e.g., no more than 0.2 bar.
      Embodiment 22. The process of any of embodiments 1-21, wherein the first pressure reduction provides a pressure inside the dehydrogenation reactor of at least 0.05 bar, e.g., at least 0.1 bar.
      Embodiment 23. The process of any of embodiments 1-22, wherein the first pressure reduction is performed for a time of at least 20 seconds, e.g., at least 30 seconds or at least 40 seconds.
      Embodiment 24. The process of any of embodiments 1-23, wherein the first pressure reduction is performed for a time in the range of 20-90 seconds, e.g., 20-75 seconds, or 20-60 seconds, or 30-90 seconds, or 30-75 seconds, or 30-60 seconds, or 40-90 seconds, or 40-75 seconds, or 40-60 seconds.
      Embodiment 25. The process of any of embodiments 1-24, wherein the first pressure reduction is performed after the flow of the first purge gas.
      Embodiment 26. The process of any of embodiments 1-25, wherein the first pressure reduction overlaps with the flow of the first purge gas, e.g., entirely overlaps.
      Embodiment 27. The process of any of embodiments 1-26, wherein the reducing stream of the reducing step further comprises hydrocarbons.
      Embodiment 28. The process of any of embodiments 1-27, wherein the reducing stream of the reducing step substantially comprises hydrogen gas (e.g., at least 80 vol %, at least 85 vol %, at least 90 vol %, or at least 95 vol %).
      Embodiment 29. The process of any of embodiments 1-28, wherein at least part of the reduction is conducted at at least 0.9 bar of hydrogen partial pressure, e.g., at least 1 bar, or at least 1.1 bar, or at least 1.2 bar.
      Embodiment 30. The process of any of embodiments 1-29, wherein at least part of the reducing step is performed at a pressure in the dehydrogenation reactor of at least 1.1 bar, e.g., at least 1.3 bar, or at least 1.5 bar.
      Embodiment 31. The process of any of embodiments 1-29, wherein at least part of the reducing step is performed at a pressure in the dehydrogenation reactor in the range of in the range of 1.1-2 bar, e.g., 1.1-1.8 bar, or 1.3-2 bar, or 1.3-1.8 bar, or 1.5-2 bar, or 1.5-1.8 bar.
      Embodiment 32. The process of any of embodiments 1-31, wherein the amount of hydrogen present in the reducing stream decreases by at least 40% (e.g., at least 50%, at least 60%, or at least 70%), during the reducing step.
      Embodiment 33. The process of any of embodiments 1-32, wherein the reducing step results in at least 60% reduction of the gas-accessible surface area of the one or more catalyst beds, e.g. at least 70%, or at least 80%.
      Embodiment 34. The process of any of embodiments 1-33, wherein the reduction is conducted for at least 10 seconds, e.g., at least 20 seconds or at least 30 seconds.
      Embodiment 35. The process of any of embodiments 1-33, wherein at least part of the reduction (e.g., at at least 0.9 bar hydrogen partial pressure) is conducted for a time in the range of 10-120 seconds, e.g., 10-90 seconds, or 20-120 seconds, or 20-90 seconds, or 30-120 seconds, or 30-90 seconds.
      Embodiment 36. The process of any of embodiments 1-35, wherein the reducing step further comprises a second pressure reduction (e.g., to reduce the pressure in the dehydrogenation reactor to no more than 0.5 bar, e.g., no more than 0.4 bar, or no more than 0.3 bar.
      Embodiment 37. The process of embodiment 35 or embodiment 36, wherein the second pressure reduction provides a pressure inside the dehydrogenation reactor of at least 0.1 bar.
      Embodiment 38. The process of any of embodiments 35-37, wherein the second pressure reduction begins at a pressure inside the dehydrogenation reactor of at least 1.1 bar (e.g., at least 1.3 bar, or at least 1.5 bar) and provides a pressure no more than 0.3 bar (e.g., no more than 0.2 bar).
      Embodiment 39. The process of any of embodiments 36-38, wherein the second pressure reduction is performed for at least 10 seconds, e.g., at least 20 seconds, or at least 30 seconds.
      Embodiment 40. The process of any of embodiments 36-38, wherein the second pressure reduction is performed for a time in the range of 10-90 seconds, e.g., 10-75 seconds, or 10-60 seconds, or 20-90 seconds, or 20-75 seconds, or 20-60 seconds, or 30-90 seconds, or 30-75 seconds, or 30-60 seconds.
      Embodiment 41. The process of any of embodiments 1-40, wherein the duration of the reduction (including any second pressure reduction) is no more than 300 seconds (e.g. no more than 240 seconds, no more than 180 seconds).
      Embodiment 42. The process of any of embodiments 1-41, wherein the duration of the reduction (including any second pressure reduction) is at least 30 seconds, e.g., at least 60 seconds.
      Embodiment 43. The process of any of embodiments 1-40, wherein the duration of the reduction (including any second pressure reduction) is in the range of 30-300 seconds, e.g., in the range of 60-300 seconds, or 30-240 seconds, or 60-240 seconds, or 30-180 seconds, or 60-180 seconds
      Embodiment 44. The process of any of embodiments 1-43, wherein one or more of the catalyst beds further comprises a heat-generating material, wherein at least one of the reducing and/or regenerating steps of the cycle causes the heat-generating material to generate heat.
      Embodiment 45. The process of any of embodiments 1-43, wherein one or more of (e.g., each of) the one or more catalyst beds includes heat-generating material in an amount that is at least 40 wt % of the combined amount of heat-generating material and dehydrogenation catalyst, e.g., at least 45 wt %, or at least 50 wt %.
      Embodiment 46. The process of any of embodiments 1-43, wherein one or more of (e.g., each of) the one or more catalyst beds includes heat-generating material in an amount in the range of 40-70 wt % of the combined amount of heat-generating material and dehydrogenation catalyst, e.g., 40-65 wt %, or 40-60 wt %, or 40-55 wt %, or 45-70 wt %, or 45-65 wt %, or 45-60 wt %, or 50-70 wt %, or 50-65 wt %.
      Embodiment 47. A system for the dehydrogenation of hydrocarbons, the system comprising:
a dehydrogenation reactor comprising one or more catalyst beds, each of the one or more catalyst beds comprising a dehydrogenation catalyst, and optionally a granular inert material and/or a heat-generating material, the heat-generating material being configured to generate heat in response to at least one of a reducing of the catalyst bed in which it is disposed and regeneration of the catalyst bed in which it is disposed;
a source of a reducing gas stream comprising hydrogen, operatively coupled to the one or more catalyst beds; and
a source of an alkane stream, operatively coupled to the at least one catalyst bed and configured to flow from an upstream direction to a downstream direction of the dehydrogenation reactor; and
a source of an oxygen-containing stream operatively coupled to the one or more catalyst beds, through an operative coupling to the dehydrogenation reactor;
a heater (e.g., an organic burning heater) capable of emitting a first purge gas (e.g., a flue gas), operatively coupled to the one or more catalyst beds, through an operative coupling to the dehydrogenation reactor;
a vacuum pump, operatively coupled to the one or more catalyst beds, through an operative coupling to the dehydrogenation reactor.
   Embodiment 48. The system of embodiment 47, wherein the heater (e.g., an organic-burning heater) is configured both to heat the alkane stream and to provide a first purge gas to purge the one or more catalyst beds.
   Embodiment 49. The system of embodiment 47 or embodiment 48, configured to perform a process according to any of embodiments 1-46.
   Embodiment 50. The process or system of any of embodiments 1-49, wherein the alkane stream comprises at least 50 wt % C₂-C₆ alkanes, as a percentage to total hydrocarbons (e.g., at least 60 wt %, or at least 75 wt %, or at least 85 wt %, or at least 90 wt %).
   Embodiment 51. The process or system of any of embodiments 1-50, wherein the alkane stream comprises at least 50 wt % C₂-C₄ alkanes, as a percentage to total hydrocarbons (e.g., at least 60 wt %, or at least 75 wt %, or at least 85 wt %, or at least 90 wt %).
   Embodiment 52. The process or system of any of embodiments 1-51, wherein the alkane stream comprises less than 10 wt % (e.g., less than 5 wt %, or less than 1 wt %) of high-coking hydrocarbons.
   Embodiment 53. The process or system of any of embodiments 1-52, wherein the dehydrogenation catalyst of the one or more catalyst beds comprises chromium, e.g., in the form of chromium oxide.
   Embodiment 54. The process or system of embodiment 53, wherein the chromium oxide is present in an amount of 5 wt % to 40 wt % of the dehydrogenation catalyst, calculated as Cr₂O₃.
   Embodiment 55. The process or system of any of embodiments 1-54, wherein the dehydrogenation catalyst comprises an alumina support.
   Embodiment 56. The process or system of any of embodiments 1-55, wherein the heat-generating material (if present) comprises a metal at a concentration of 2 wt % to 40 wt % of the total heat-generating material.
   Embodiment 57. The process or system of embodiment 56, wherein the metal comprises at least one of copper, chromium, molybdenum, vanadium, cerium, yttrium, scandium, tungsten, manganese, iron, cobalt, nickel, silver, or bismuth.
   Embodiment 58. The process or system of any of embodiments 1-57, wherein the heat-generating material (if present) comprises a promoter, wherein the promoter comprises an alkali metal, an alkaline earth metal, or zirconium.
   Embodiment 59. The process or system of any of embodiments 1-58, wherein the granular inert material is present in the one or more catalyst beds.
   Embodiment 60. The process or system of any of embodiments 1-49, wherein the granular inert material is a granular alumina, such as granular alpha-alumina.
   Embodiment 61. The process or system of any of embodiments 1-60, wherein the granular inert material is a mixture of silica and alumina, or zirconia and alumina, or titania and alumina, in any proportions.
   Embodiment 62. The process or system of any of embodiments 1-61, wherein the one or more catalyst beds operate in a cyclic redox mode.
   Embodiment 63. The process or system of any of embodiments 1-62, wherein the dehydrogenation reactor operates at a temperature in the range of 500-1050° C., or 500-1000° C., or 500-950° C., or 500-900° C., or 500-850° C., or 500-800° C., or 500-750° C., or 500-700° C., or 500-650° C., or 500-600° C.
   Embodiment 64. The process or system of any of embodiments 1-62 wherein the dehydrogenation reactor operates at a temperature in the range of 500-700° C., or 520-700° C., or 540-700° C., or 560-700° C., or 580-700° C., or 600-700° C., or 500-680° C., or 500-660° C., or 500-640° C., or 500-620° C., or 500-600° C., or 520-680° C.
   Embodiment 65. The process or system of any of embodiments 1-62 wherein the dehydrogenation step is performed at a pressure in the range of 0.05-3 bar, e.g., 0.1-3 bar, or 0.2-3 bar, or 0.05-2 bar, or 0.1-2 bar, or 0.2-2 bar.
   Embodiment 66. The process or system of any of embodiments 1-65, wherein the alkane stream is supplied to the one or more catalyst beds at a liquid hourly space velocity of 0.1-4 per hour, e.g., 0.1-3 per hour, or 0.5-4 per hour, or 0.5-3 per hour, or 1-4 per hour, or 1-3 per hour.

The particulars shown herein are by way of example and for purposes of illustrative discussion of certain embodiments of the present disclosure only and are presented in the cause of providing what is believed to be the most useful and readily understood description of the principles and conceptual aspects of various embodiments of the disclosure. In this regard, no attempt is made to show details associated with the methods of the disclosure in more detail than is necessary for the fundamental understanding of the methods described herein, the description taken with the examples making apparent to those skilled in the art how the several forms of the methods of the disclosure may be embodied in practice. Thus, before the disclosed processes and devices are described, it is to be understood that the aspects described herein are not limited to specific embodiments, apparatus, or configurations, and as such can, of course, vary. It is also to be understood that the terminology used herein is for the purpose of describing particular aspects only and, unless specifically defined herein, is not intended to be limiting.

The terms “a,” “an,” “the” and similar referents used in the context of describing the methods of the disclosure (especially in the context of the embodiments and claims described herein) are to be construed to cover both the singular and the plural, unless otherwise indicated herein or clearly contradicted by context.

All percentages, ratios and proportions herein are by weight, unless otherwise specified.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

Some embodiments of various aspects of the disclosure are described herein, including the best mode known to the inventors for carrying out the methods described herein. Of course, variations on these described embodiments will become apparent to those of ordinary skill in the art upon reading the foregoing description. The skilled artisan will employ such variations as appropriate, and as such the methods of the disclosure can be practiced otherwise than specifically described herein. Accordingly, the scope of the disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

The phrase “at least a portion” as used herein is used to signify that, at least, a fractional amount is required, up to the entire possible amount.

In closing, it is to be understood that the various embodiments herein are illustrative of the methods of the disclosures. Other modifications that may be employed are within the scope of the disclosure. Thus, by way of example, but not of limitation, alternative configurations of the methods may be utilized in accordance with the teachings herein. Accordingly, the methods of the present disclosure are not limited to that precisely as shown and described.

1. A process for the dehydrogenation of hydrocarbons, the process comprising:
providing a dehydrogenation reactor comprising one or more catalyst beds, each of the one or more catalyst beds comprising a dehydrogenation catalyst, and, optionally, a granular inert material; and
performing the following cycle of steps a plurality of times:
regenerating the one or more catalyst beds by contacting the one or more catalyst beds with an oxygen-containing stream; then
purging the one or more catalyst beds by flowing therethrough a first purge gas stream having no more than 5 vol % O₂ and in a first pressure reduction reducing the pressure in the dehydrogenation reactor to no more than 0.5 bar; then
reducing the one or more catalyst beds by flowing therethrough a reducing stream comprising hydrogen, at least part of the reduction being conducted at at least 0.9 bar of hydrogen partial pressure; then
contacting the one or more catalyst beds with an alkane stream by flowing from an upstream direction to a downstream direction, to dehydrogenate the alkane stream to provide an alkene stream, the dehydrogenation being endothermic; then
purging the one or more catalyst beds with a second purge gas stream to substantially remove hydrocarbon therefrom.

2. The process of claim 1, wherein the purging and pressure reduction are performed to remove at least 90% of the oxygen from the catalyst bed.

3. The process of claim 1, wherein the first purge gas stream comprises no more than 3 vol % oxygen gas.

4. The process of claim 1, wherein the first purge gas stream comprises 1-5 vol % oxygen gas.

5. The process of claim 1, wherein the first purge gas stream comprises no more than 2 vol % hydrogen.

6. The process of claim 1, wherein the first purge gas comprises at least 50 vol % nitrogen gas.

7. The process of claim 1, wherein first purge gas comprises a flue gas that comprises products of combustion of hydrocarbons.

8. The process of claim 7, wherein the flue gas is provided by an organic-burning heater having a flue output operatively coupled to the one or more catalyst beds.

9. The process of claim 8, further comprising heating the alkane stream with the organic-burning heater.

10. The process of claim 1 wherein the flowing of the first purge gas stream is performed for a time of at least 30 seconds.

11. The process of claim 1, wherein the dehydrogenation catalyst of the one or more catalyst beds comprises chromium, e.g., in the form of chromium oxide.

12. The process of claim 1, wherein the dehydrogenation reactor operates at a temperature in the range of 500-700° C.

13. The process or system of claim 1, wherein the dehydrogenation step is performed at a pressure in the range of 0.2-2 bar.

14. A system for the dehydrogenation of hydrocarbons, the system comprising:
a dehydrogenation reactor comprising one or more catalyst beds, each of the one or more catalyst beds comprising a dehydrogenation catalyst, and optionally a granular inert material and/or a heat-generating material, the heat-generating material being configured to generate heat in response to at least one of a reducing of the catalyst bed in which it is disposed and regeneration of the catalyst bed in which it is disposed;
a source of a reducing gas stream comprising hydrogen, operatively coupled to the one or more catalyst beds; and
a source of an alkane stream, operatively coupled to the at least one catalyst bed and configured to flow from an upstream direction to a downstream direction of the dehydrogenation reactor; and
a source of an oxygen-containing stream operatively coupled to the one or more catalyst beds, through an operative coupling to the dehydrogenation reactor;
a heater (e.g., an organic burning heater) capable of emitting a first purge gas (e.g., a flue gas), operatively coupled to the one or more catalyst beds, through an operative coupling to the dehydrogenation reactor;
a vacuum pump, operatively coupled to the one or more catalyst beds, through an operative coupling to the dehydrogenation reactor.

15. The system of claim 14, wherein the heater (e.g., an organic burning heater) is configured both to heat the alkane stream and to provide a first purge gas to purge the one or more catalyst beds.

16. The system of claim 14, wherein the heater is an organic burning heater.

17. The system of claim 14, wherein the first purge gas is a flue gas.

18. The method of claim 1, wherein the second purge gas stream is a steam stream.
